(19) United States
(12) Patent Application Publication
(10) Pub. No.: US 2010/0068265 A1
(43) Pub. Date: Mar. 18, 2010

(54) GELATIN CAPSULES COMPRISING AN ACID

(76) Inventors: Isabel Ottinger, Feiburg (DE); Agnes Taillardat, Seewen (CH)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 12/518,958

(22) PCT Filed: Dec. 18, 2007
(86) PCT No.: PCT/EP2007/011138

§ 371 Date: Jul. 21, 2009

(30) Foreign Application Priority Data
Dec. 20, 2006 (EP) 06126698.7

(51) Int. Cl. A61K 9/64; A61K 31/438; A61P 35/00

(52) U.S. Cl. 424/456; 514/283

(57) ABSTRACT

A pharmaceutical composition in the form of a gelatin capsule comprising a pharmaceutically acceptable acid.

The present invention relates to gelatin capsules. Such capsules may be used in, inter alia, the pharmaceutical, nutraceutical, and food industries.

Gelatin, a mixture of water-soluble proteins derived from collagen by hydrolysis, is widely used in the pharmaceutical and food industries. One major application of gelatin is in preparation of both hard and soft gelatin capsules. Gelatin capsules show a potential to form pellicles. This pellicle formation may be a cross-linking of the gelatin making it partially insoluble in water. Pellicle formation may be induced by the exposure of gelatin to high humidity, heat or trace of reactive chemical agents such as aldehydes. Pellicle formation may affect the dissolution of a drug e.g. exhibiting a drop in the dissolution rate. This drop in dissolution rate may lead to undesirable and unacceptable alterations in vitro dissolution profile and in bioavailability, especially for drugs of low water solubility or drugs whose absorption is dissolution-rate limited.

US patent application 2004/0105885 describes compositions suitable for the preparation of capsule shells comprising gelatin and at least one sulfite compound present in an amount effective to inhibit cross-linking of the gelatin shell and/or pellicle formation upon storage. US patent application 2004/0105883 discloses a pharmaceutical dosage form comprising a fill material sealed in capsule shells wherein the fill material comprises a selective cyclooxygenase-2 inhibitory drug of low water solubility and an amine agent comprising at least one pharmaceutically acceptable primary or secondary amine, wherein the capsule shells comprise gelatin, and wherein the amine agent is present in an amount sufficient to inhibit pellicle formation in the capsule shells upon storage of the dosage form. U.S. Pat. No. 5,874,106 describes a method of reducing crosslinking in gelatin capsules wherein an amino acid and a carboxylic acid are incorporated into the capsule fill.

There is a difficulty to dose some of the compounds inhibiting pellicle formation described in the prior art. If compounds such as sulfite compounds or amines, e.g. nitrosamines, are not dosed very carefully they might have toxic effects and cannot be used for pharmaceuticals.

Surprisingly the inventors of the present invention have found that presence of pharmaceutically acceptable acids will be sufficient to prevent pellicle formation of a gelatin capsule. Therefore the present invention provides a pharmaceutical composition in the form of a gelatin capsule comprising a pharmaceutically acceptable acid. The pharmaceutical composition may comprise a capsule shell and fill material. The pharmaceutically acceptable acid may be added to the capsule shell or to the fill material or to both the capsule shell and the fill material. The pharmaceutically acceptable acid may migrate from the capsule shell to the fill material or from the fill material to the capsule shell hereby achieving the desired effect. The migration of the pharmaceutically acceptable acid has the advantage that it may be added to the fill material or to the gelatin shell or to both with the effect that it will be distributed everywhere in the pharmaceutical composition in the form of a gelatin capsule. Thereby the pharmaceutically acceptable acid may effectively inhibit pellicle formation. Preferably the pharmaceutically acceptable acid is added to the fill material.

The pharmaceutically acceptable acid may be selected from the group including fumaric acid, phosphoric acid, e.g. diphosphoric acid, maleic acid, ascorbic acid, tartaric acid, malonic acid, glucuronic acid and citric acid. According to the present invention preferably citric acid and phosphoric acid may be used.

The pharmaceutically acceptable acid may be present in an amount effective to inhibit pellicle formation of the composition in form of a gelatin capsule. The pharmaceutically acceptable acid may be present in an amount of about 0.01% to about 20%, e.g. of about 0.05% to about 10%, e.g. of about 0.075% to about 5%, e.g. of about 0.1% to about 2% of the total weight of the capsule shell. In another aspect the pharmaceutically acceptable acid may be present in amount of about 0.01% to about 20%, e.g., of about 0.05% to about 10%, e.g. of about 0.075% to about 5%, e.g. of about 0.1% to about 2% of the total weight of the fill material.

Pharmaceutically acceptable acids, e.g. citric acid, have the advantage that pellicle formation of the gelatin capsule may be inhibited without causing any toxicological effects. With pharmaceutically acceptable acids a reduction of the dissolution rate impairing the achievable shelf life and/or a reduced bioavailability of a pharmaceutically active agent may be prevented. Accordingly, pharmaceutical compositions of the invention comprising pharmaceutically acceptable acids as defined above may show advantageous safety and improved efficacy.

In another aspect of the invention the composition does not contain an amino acid. Further the composition does not contain any other compound for prevention of pellicle formation than the pharmaceutically acceptable acid. According to the present invention pellicle formation may be prevented by a pharmaceutically acceptable acid, e.g. citric acid. Of course, besides the pharmaceutically acceptable acid the composition may contain further excipients.

The term “pellicle” herein refers to a relatively water-insoluble membrane formed in a gelatin capsule shell wherein the membrane tends to be thin, tough, and rubbery. One mechanism underlying pellicle formation is gelatin cross-linking resulting in partially insoluble gelatin and a reduced dissolution rate.

A composition of the present invention may further comprise in addition to gelatin and a pharmaceutically acceptable acid a pharmaceutically active agent. The pharmaceutically active agent may be chosen from therapeutic compounds which include antacids, anti-inflammatory substances, coronary dilators, cerebral dilators, peripheral vasodilators, anti-infectives, psychotropics, antiaminics, stimulants, antihistamines, anti-cancer therapeutic compounds, laxatives, decongestants, vitamins, gastrointestinal sedatives, antidiarrheal preparations, anti-anginal therapeutic compounds, vasodilators, antiarrythmics, anti-hypertensive therapeutic compounds, vasoconstrictors and migraine treatments, anticoagulants and antithrombotic therapeutic compounds, analgesics, anti-pyretics, hypnotics, sedatives, anti-emetics, anti-nauseants, anti-convulsants, neuromuscular therapeutic compounds, hyper- and hypoglycemic agents, thyroid and anti-thyroid preparations, diuretics, anti-spasmodics, uterine relaxants, mineral and nutritional additives, anti-obesity therapeutic compounds, anabolic therapeutic compounds, erythropoietic therapeutic compounds, anti-asthmatics, expectorants, cough suppressants, mucolytics, and anti-uricemic therapeutic compounds.

A preferred active agent is an inhibitor of topoisomerase I (Topo I inhibitor) and is therefore capable of preventing disease symptoms that are caused inter alia by the activation of the topoisomerase I receptor.

More specifically a preferred active agent is a camptothecin derivative. This class of compounds is described in U.S. Pat. No. 6,242,457.

Preferred active agents, which are described in U.S. Pat. No. 6,242,457, include:
7-methyoxyiminomethylcamptothecin;
7-methoxyiminomethyl-10-hydroxycamptothecin;
7-(ter-butoxycarbonyl-2-propoxy)iminomethylcamptothecin;
7-ethoxyiminomethylcamptothecin;
7-isopropoxyiminomethylcamptothecin;
7-(2-methylbutoxy)iminomethylcamptothecin;
7-t-butoxyiminomethylcamptothecin;
7-t-butoxyiminomethyl-10-hydroxycamptothecin;
7-t-butoxyiminomethyl-10-methoxycamptothecin;
7-(4-hydroxybutoxy)iminomethylcamptothecin;
7-triphenylmethoxyiminomethylcamptothecin;
7-carboxymethoxyiminomethylcamptothecin;
7-(2-amino)ethoxyiminomethylcamptothecin;
7-(2-N,N-dimethylamino)ethoxyiminomethylcamptothecin;
7-allyloxyiminomethylcamptothecin;
7-cyclohexyloxyiminoethylcamptothecin;
7-cyclohexylmethoxyiminomethylcamptothecin;
7-cyclooctyloxyiminomethylcamptothecin;
7-cyclooctylmethoxyiminomethylcamptothecin;
7-benzyloxyiminomethylcamptothecin;
7-[(1-benzyloxyimino)-2-phenylethyl]camptothecin;
7-(1-benzyloxyimino)ethylcamptothecin;
7-phenoxyiminomethylcamptothecin;
7-(1-t-butoxyimino)ethylcamptothecin;
7-p-nitrobenzyloxyiminomethylcamptothecin;
7-p-methylbenzyloxyiminomethylcamptothecin;
7-pentafluorobenzyloxyiminomethylcamptothecin;
7-p-phenylbenzyloxyiminomethylcamptothecin;
7-[2-(2,4-difluorophenyl)ethoxy]iminomethylcamptothecin;
7-(4-t-butylbenzyloxy)iminomethylcamptothecin;
7-(1-adamantyloxy)iminomethylcamptothecin;
7-(1-adamantylmethoxy)iminomethylcamptothecin;
7-(2-naphthyloxy)iminomethylcamptothecin;
7-(9-anthrylmethoxy)iminomethylcamptothecin;
7-oxiranylmethoxyiminomethylcamptothecin;
7-(6-uracyl)methoxyiminomethylcamptothecin;
7-[2-(1-urcyl)ethoxy]iminomethylcamptothecin;
7-(4-pyridyl)methoxyiminomethylcamptothecin;
7-(2-thienyl)methoxyiminomethylcamptothecin;
7-[(N-methyl)-4-piperidinyl]methoxyiminomethylcamptothecin;
7-[2-(4-morpholininyl]ethoxy]iminomethylcamptothecin;
7-(benzoyloxyiminomethyl)camptothecin;
7-[(1-hydroxyimino)-2-phenylethyl)camptothecin;
7-ter-butyloxyiminomethylcamptothecin-N-oxide; and
7-methoxyiminomethylcamptothecin N-oxide.

In a very preferred embodiment of the invention, the topoisomerase I inhibitor of formula I has the following structure known as Compound A:

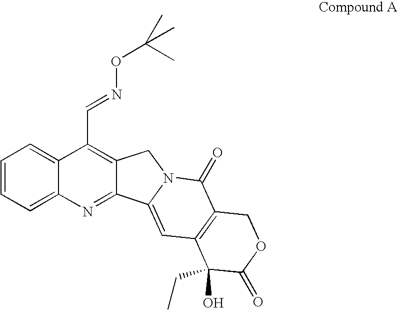

The preferred and especially preferred active agents, in free or pharmaceutically acceptable salt form, may be prepared as described in U.S. Pat. No. 6,424,457. As mentioned therein, they may be in the form of their possible enantiomers, diastereoisomers and relative mixtures, the pharmaceutically acceptable salts thereof and their active metabolites.

In accordance with the present invention the active agent may be present in an amount by weight of up to about 20% by weight of the composition of the invention, e.g. from about 0.05% by weight. The active agent is preferably present in an amount of 0.5 to 15% by weight of the composition.

In addition to prevent pellicle formation, pharmaceutically acceptable acids may stabilize compothecin derivatives. Free protons of the acids may stabilize the lacton ring of these molecules. 7-t-butoxyiminomethyl-campothecin may be stabilized while the acids protanate the N-atom in the Naphty-ring of the molecule preventing its oxidation as well as stabilizing the lacton ring. Thus, the use of pharmaceutically acceptable acid may have several beneficial effects in campothecin derivates.

The composition of the invention may further comprise one or more pharmaceutically acceptable excipients.

For the preparation of soft gelatin capsules the composition may comprise at least one plasticizer. The plasticizer may be present in an amount of about 5% to about 50%, preferably about 10% to about 30% of the total weight of the composition. Examples of suitable plasticizers include poly-hydroxy-alcohols, e.g. sorbitol, glycerol or mannitol, dialkylphtalates, glycols and polyglycols including polyethylene glycols with a molecular weight of about 200 to about 40000, methoxyl-propylene-glycol, and 1,2 propylene glycole, esters of polyhydroxy-alcohols such as mono-, di-, and tri-acetate of glycerol, ricinoelic acid and esters thereof, and mixtures of the above plasticizers.

In a preferred embodiment of the present invention at least about 40%, preferably at least about 50%, still more preferably at least about 60% of the pharmaceutically acceptable acid present in a dosage form of the invention is present in the fill material. The fill material comprising the active agent and the pharmaceutically acceptable acid may be in the form of a semi-solid or liquid. The pharmaceutically acceptable acid of the semi-solid or liquid fill material may migrate into the shell and inhibit pellicle formation of the gelatin capsule.

The composition of the fill material of the present invention may be one or more lipophilic excipients, one or more hydrophilic excipients, one or more surfactants or mixtures thereof comprising an active agent. In a preferred embodiment, the composition of the fill material is a spontaneously dispersible composition comprising an active agent. The composition may most preferably be a microemulsion preconcentrate.

Terms used in the specification have the following meaning:

“Spontaneously dispersible pharmaceutical composition” as used herein means a composition that contains an active agent herein defined and is capable of producing colloidal structures-when diluted with an aqueous medium, for example water, or in gastric juices. The colloidal structures are preferably liquid droplets in the microemulsion size range. Solid drug particles, either crystalline or amorphous, of mean diameter greater than 200 nm may also be present. The spontaneously dispersible pharmaceutical composition is preferably a microemulsion preconcentrate.
“Microemulsion preconcentrate” as used herein means a composition which spontaneously forms a microemulsion in an aqueous medium, for example, in water, for example on dilution of 1:1 to 1:300, preferably 1:1 to 1:70, but especially 1:1 to 1:10 or in the gastric juices after oral application.
“Microemulsion” as used herein means a translucent, slightly opaque, opalescent, non-opaque or substantially non-opaque colloidal dispersion that is formed spontaneously or substantially spontaneously when its components are brought into contact with an aqueous medium. A microemulsion is thermodynamically stable and typically contains dispersed droplets of a mean diameter less than about 200 nm (2000 Å). Generally microemulsions comprise droplets or liquid nanoparticles that have a mean diameter of less than about 150 nm (1500 Å); typically less than 100 nm, generally greater than 10 nm, and they are stable over periods up to 24 hours or longer.

In another aspect, the fill of the present invention provides a spontaneously dispersible pharmaceutical composition comprising a camptothecin derivative, and a carrier medium comprising a lipophilic component, a surfactant, a hydrophilic component and optionally a co-solvent.

Preferably the spontaneously dispersible pharmaceutical composition is suitable for oral administration.

The camptothecin derivatives may have poor water solubility characteristics and may display a water solubility of below 0.001%, e.g. 0.001 to 0.0001%.

The active agent is preferably used in free base form.

In another aspect, the fill of the present invention provides a microemulsion preconcentrate comprising a camptothecin derivative. Especially interesting is that the drug load achieved within the microemulsion pre-concentrates is significantly higher than within the single excipients indicating an over-additive solubility of the camptothecin derivatives within the microemulsion preconcentrates.

In a further aspect, the fill of the present invention provides a microemulsion preconcentrate comprising a camptothecin derivative and a carrier medium that comprises a lipophilic component, a surfactant, a hydrophilic component and a optionally co-solvent.

The microemulsion preconcentrate preferably forms an o/w (oil-in-water) microemulsion when diluted with water.

Preferably the relative proportions of the lipophilic component(s), the surfactant(s), the hydrophilic component(s), and optionally the co-solvent(s) lie within the “Microemulsion” region on a standard three way plot graph. These phase diagrams, can be generated in a conventional manner as described in e.g. GB 2,222,770 or WO 96/13273.

In another aspect, the fill of the present invention provides a microemulsion comprising a camptothecin derivative.

The microemulsion is preferably an o/w (oil-in-water) microemulsion.

In another aspect, the fill of the present invention provides a microemulsion comprising a camptothecin derivatives, a lipophilic component, a surfactant, water, a hydrophilic component and optionally a co-solvent The colloidal structures of the microemulsion form spontaneously or substantially spontaneously when the components of the composition of the invention are brought into contact with an aqueous medium, e.g. by simple shaking by hand for a short period of time, for example for 10 seconds. The compositions of the invention are thermodynamically stable, e.g. for at least 15 minutes or up to 4 hours, even to 24 hours or longer. Typically, they contain dispersed structures, i.e. droplets or liquid nanoparticles of a mean diameter less than about 200 nm (2,000 Å), e.g. less than about 150 nm (1,500 Å), typically less than 100 nm (1,000 Å), generally greater than 10 nm (100 Å) as measured by standard light scattering techniques, e.g. using a MALVERN ZETASIZER 3000™ particle characterising machine. Solid drug particles of mean diameter greater than 200 nm may also be present. The proportion of particles present may be temperature dependent.

The active agent is poorly water soluble so it is carried in a carrier medium.

In some embodiments of the compositions of the fill of the invention the carrier medium comprises a lipophilic component, a surfactant, and a hydrophilic component. In other embodiments the carrier medium comprises a lipophilic component, a surfactant, a hydrophilic component and a co-solvent.

Fill material of the present invention may further comprise at least one pharmaceutically acceptable antioxidant. Examples of antioxidants include alpha-tocopherol (vitamin E), ascorbic acid (vitamin C), and salts thereof including sodium ascorbate and ascorbic acid palmitate, butylated hydroxyanisole (BHA), butylated hydroxytoluene (BHT), fumaric acid and salts thereof, hypophosphorous acid, malic acid, alkyl gallates, e.g., propyl gallate, octyl gallate and lauryl gallate, sodium sulfite, sodium bisulfite and sodium metasulfite.

The antioxidant may be present in a dosage form of the invention in amount of about 0.05% to about 1% of the total weight of the fill material.

Fill material of the present invention may optionally comprise one or more pharmaceutically acceptable sweetener. Examples of sweeteners or flavoring agents typically provide up to 2.5 or 5% by weight based on the total weight of the composition. Sweetners include mannitol, propylene glycol, sodium saccharine, neotame and aspartame.

Fill material of the present invention may further comprise one or more sedimentation inhibitors which enhances the viscosity of the fill material. Examples of the sedimentation inhibitor of the present invention include but are not limited to precipitated or colloidal silica, e.g., Aerosil® (loc. cit. H. Fiedler “Lexikon der Hllfsstoffe”, 5^{th} Edition, ECV Aulendorf 2002, volume 1, page 158), Bentonit, and zinc/aluminium stearate.

The lipophilic component comprises one or more lipophilic substances. The hydrophilic component comprises one or more hydrophilic substances. The carrier medium can contain one or more surfactants. The carrier medium can contain one or more co-solvents.

The compositions of the fill of the invention may include a lipophilic component. The active agent may be contained in this component of the carrier medium. The lipophilic component (when present) is preferably characterized by a low HLB value of less than 10, e.g. up to 8.

Suitable lipophilic components include:

1) Glyceryl mono-C₆-C₁₄-Fatty Acid Esters
These are obtained esterifying glycerol with vegetable oil followed by molecular distillation. Monoglycerides suitable for use in the compositions of the invention include both symmetric (i.e. β-monoglycerides) as well as asymmetric monoglycerides (α-monoglycerides. They also include both uniform glycerides (in which the fatty acid constituent is composed primarily of a single fatty acid) as well as mixed glycerides (i.e. in which the fatty acid constituent is composed of various fatty acids) The fatty acid constituent may include both saturated and unsaturated fatty acids having a chain length of from e.g. C₈-C₁₄. Particularly suitable are caprylic or lauric acid monoglycerides which are commercially available, e.g. under the trade names Imwitor® 308 or Imwitor® 312, respectively, from e.g. sasol. For example Imwitor® 308 comprises at least 80% monoglycerides and exhibits the following additional characterising data: free glycerol max 6%, acid value max. 3, saponification value 245-265, iodine value max. 1, water content max. 1%. Typically it comprises 1% free glycerol, 90% monoglycerides, 7% diglycerides, 1% triglycerides (H. Fiedler, loc. cit., volume 1, page 906). A further example is Capmul MCM C8 from Abitec Corporation.
   2) Mixtures of Mono- and di-glycerides of C₆-C₁₈ Fatty Acids
These include both symmetric (i.e. β-monoglycerides and α,α¹-diglycerides) as well as asymmetric mono- and di-glycerides (i.e. α-monoglycerides and α,β-diglycerides) and acetylated derivatives thereof. They also include both uniform glycerides (in which the fatty acid constituent is composed primarily of a single fatty acid) as well as mixed glycerides (i.e. in which the fatty acid constituent is composed of various fatty acids) and any derivatives thereof with lactic or citric acid. The fatty acid constituent may include both saturated and unsaturated fatty acids having a chain length of from e.g. C₈-C₁₀. Particularly suitable are mixed caprylic and capric acid mono- and di-glycerides as commercially available, e.g. under the trade name Imwitor® 742 or Imwitor 928, from e.g. sasol. For example Imwitor® 742 comprises at least 45% monoglycerides and exhibits the following additional characterising data: free glycerol max. 2%, acid value max. 2, saponification value 250-280, iodine value max. 1, water max. 2% (H. Fiedler, loc. cit., vol 1, page 906). Other suitable mixtures comprise mono/diglycerides of caprylic/capric acid in glycerol as known and commercially available under e.g. the trade name Capmul® MCM from e.g. Abitec Corporation. Capmul® MCM exhibits the following additional characterising data: acid value 2.5 max., alpha-Mono (as oleate) 80% min., free glycerol 2.5% max., iodine value 1 max., chain length distribution: caproic acid (C6) 3% max., caprylic acid (C8) 75% min., capric acid (C10) 10% min., lauric acid (C12) 1.5% max., moisture (by Karl Fisher) 0.5% max. (manufacturer information). Suitable examples of mono-/di-glcyerides with additional derivatization with lactic or citric acid are those marketed under the brand names of Imwitor 375, 377 or 380 by sasol. Furthermore, the fatty acid constituent may include both saturated and unsaturated fatty acids having a chain length of from e.g. C₁₆-C₁₈. A suitable example is Tegin® O (glyceryl oleate) exhibiting the following additional characterising data: monoglyceride content 55-65%, peroxide value max. 10, water content max. 1%, acid value max. 2, iodine value 70-76, saponification value 158-175, free glycerol max. 2%, (manufacturer information).
   3) Glyceryl di-C₆-C₁₈-Fatty Acid Esters
These include symmetric (i.e. α,α¹-diglycerides) and asymmetric diglycerides (i.e. α,β-diglycerides) and acetylated derivatives thereof. They also include both uniform glycerides (in which the fatty acid constituent is composed primarily of a single fatty acid) as well as mixed glycerides (i.e. in which the fatty acid constituent is composed of various fatty acids) and any acetylated derivatives thereof. The fatty acid constituent can include both saturated and unsaturated fatty acids having a chain length of from C₆-C₁₈, e.g. C₆-C₁₆, e.g. C₈-C₁₀, e.g. C₈. Particularly suitable is caprylic diglycerides, which is commercially available, e.g. under the trade name Sunfat® GDC-S, e.g. from Taiyo Kagaku Co., Ltd. Sunfat® GDC-S has an acid value of about 0.3, a diglyceride content of about 78.8%, and a monoester content of about 8.9.

### 4) Medium Chain Fatty Acid Triglyceride

These include triglycerides of saturated fatty acid having 6 to 12, e.g. 8 to 10, carbon atoms. Suitable medium chain fatty acid triglycerides are those known and commercially available under the trade names Acomed®, Myritol®, Captex®, Neobee®M 5 F, Miglyol®810, Miglyol®812, Miglyol®818, Mazol®, Sefsol®860, Sefsol®870; Miglyol®812 being the most preferred. Miglyol®812 is a fractionated coconut oil comprising caprylic-capric acid triglycerides and having a molecular weight of about 520 Daltons. Fatty acid composition=C₆ max. about 3%, C₈ about 50 to 65%, C₁₀ about 30 to 45%, C₁₂ max 5%; acid value about 0.1; saponification value about 330 to 345; iodine value max 1. Miglyol® 812 is available from Condea. Neobee® M 5 F is a fractionated caprylic-capric acid triglyceride available from coconut oil; acid value max. 0.2; saponification value about 335 to 360; iodine value max 0.5, water content max. 0.15%, D.²⁰ 0.930-0.960, n_{D}²⁰ 1.448-1.451 (manufacturer information). Neobee®M 5 F is available from Stepan Europe. A further example is Miglyol 829 containing additionally esters with succinic acid.

### 5) Glyceryl Mono-C Acid Esters

These are obtained esterifying glycerol with vegetable oil followed by molecular distillation. Monoglycerides suitable for use in the compositions of the invention include both symmetric (i.e. β-monoglycerides) as well as asymmetric monoglycerides (α-monoglycerides. They also include both uniform glycerides (in which the fatty acid constituent is composed primarily of a single fatty acid) as well as mixed glycerides (i.e. in which the fatty acid constituent is composed of various fatty acids) The fatty acid constituent may include both saturated and unsaturated fatty acids having a chain length of from e.g. C₁₆-C₁₈. Suitable examples include GMOrphic by Eastman, Rylo MG20 distilled monoglyceride by Danisco Ingredients, or Monomuls 90-O18 by Henkel. For example GMOrphic®-80 (glyceryl monooleate) exhibits the following additional characterising data: monoglyceride content min. 94%, C18:1 content 75% min., peroxide value max. 2.5, C18:2+C18:3 max. 15%, C16:0+C18:0+C20:0 max. 10%, water max. 2%, acid value max. 3, iodine value 65-75, saponification value 155-165, free glycerine max. 1%, hydroxyl number 300-330 (manufacturer information).
   6) Mixed mono-, di-, tri-glycerides
These include mixed mono-, di-, tri-glycerides that are commercially available under the trade name Maisine® from Gattefossé. They are transesterification products of corn oil and glycerol. Such products are comprised predominantly of linoleic and oleic acid mono-, di- and tri-glycerides together with minor amounts of palmitic and stearic acid mono-, di- and tri-glycerides (corn oil itself being comprised of ca. 56% by weight linoleic acid, 30% oleic acid, ca. 10% palmitic and ca. 3% stearic acid constituents). Physical characteristics are: free glycerol max 10%, monoglycerides ca. 40%, diglycerides ca. 40%, triglycerides ca. 10%, free oleic acid content ca. 1%. Further physical characteristics are: acid value max. 2, iodine value of 85-105, saponification value of 150-175, mineral acid content=0. The fatty acid content for Maisine® is typically: palmitic acid ca. 11%, stearic acid ca. 2.5%, oleic acid ca. 29%, linoleic acid ca. 56%, others ca. 1.5% (H. Fiedler, loc. cit., volume 2, page 1079; manufacturer information).
Mixed mono-, di-, tri-glycerides preferably comprise mixtures of C₈ to C₁₀ or C₁₂₋₂₀ fatty acid mono-, di- and tri-glycerides, especially mixed C₁₆₋₁₈ fatty acid mono-, di- and triglycerides. The fatty acid component of the mixed mono-, di- and tri-glycerides may comprise both saturated and unsaturated fatty acid residues. Preferably however they are predominantly comprised of unsaturated fatty acid residues; in particular C₁₈ unsaturated fatty acid residues. Suitably the mixed mono-, di-, tri-glycerides comprise at least 60%, preferably at least 75%, more preferably at least 85% by weight of a C₁₈ unsaturated fatty acid (for example linolenic, linoleic and oleic acid) mono-, di- and tri-glycerides. Suitably the mixed mono-, di-, tri-glycerides comprise less than 20%, for example about 15% or 10% by weight or less, saturated fatty acid (for example palmitic and stearic acid) mono-, di- and tri-glycerides. Mixed mono-, di-, tri-glycerides are preferably predominantly comprised of mono- and di-glycerides; for example mono- and di-glycerides comprise at least 50%, more preferably at least 70% based on the total weight of the lipophilic phase or component. More preferably, the mono- and di-glycerides comprise at least 75% (for example about 80% or 85% by weight of the lipophilic component. Preferably monoglycerides comprise from about 25 to about 50%, based on the total weight of the lipophilic component, of the mixed mono-, di-, tri-glycerides. More preferably from about 30 to about 40% (for example 35 to 40%) monoglycerides are present. Preferably diglycerides comprise from about 30 to about 60%, based on the total weight of the lipophilic component, of the mixed mono-, di-, tri-glycerides. More preferably from about 40 to about 55% (for example 48 to 50%) diglycerides are present. Triglycerides suitably comprise at least 5% but less than about 25%, based on the total weight of the lipophilic component, of the mixed mono-, di-, tri-glycerides. More preferably from about 7.5 to about 15% (for example from about 9 to 12%) triglycerides are present. Mixed mono-, di-, tri-glycerides may be prepared by admixture of individual mono-, di- or tri-glycerides in appropriate relative proportion. Conveniently however they comprise trans-esterification products of vegetable oils, for example almond oil, ground nut oil, olive oil, peach oil, palm oil or, preferably, corn oil, sunflower oil or safflower oil and most preferably corn oil, with glycerol. Such transesterification products are generally obtained as described in GB 2 257 359 or WO 94/09211. Preferably some of the glycerol is first removed to give a “substantially glycerol free batch” when soft gelatine capsules are to be made. Purified transesterification products of corn oil and glycerol provide particularly suitable mixed mono-, di-, and tri-glycerides hereinafter referred to as “refined oil” and produced according to procedures described in United Kingdom patent specification GB 2,257,359 or international patent publication WO 94/09211.

### 7) Acetylated Monoglycerides (C18)

These include Myvacet 9-45.

### 8) Propylene Glycol Monofatty Acid Esters

The fatty acid constituent may include both saturated and unsaturated fatty acids having a chain length of from e.g. C₈-C₁₂. Particularly suitable are propylene glycol mono ester of caprylic and lauric acid as commercially available, e.g. under the trade names Sefsol® 218, Capryol®90 or Lauroglycol®90 (H. Fiedler, loc. cit., vol 2, page 1025, manufacturer information), from e.g. Nikko Chemicals Co., Ltd. or Gattefossé or Capmul PG-8 from Abitec Corporation. For example Lauroglycol®90 exhibits the following additional characterising data: acid value max. 8, saponification value 200-220, iodine value max. 5, free propylene glycol content max. 5%, monoester content min. 90%; Sefsol® 218 exhibits the following additional characterising data: acid value max. 5, hydroxy value 220-280.

### 9) Propylene Glycol Mono- and Di-Fatty Acid Esters

These include Laroglycol FCC and Capryol PGMC.

### 10) Propylene Glycol Diesters

Propylene glycol di-fatty acid esters such as propylene glycol dicaprylate (which is commercially available under the trade name Miglyol® 840 from e.g. sasol; H. Fiedler, loc. cit., volume 2, page 1130) or Captex 200 from Abitec Corporation.

### 11) Propylene Glycol Monoacetate and Propylene Glycol Diacetate

### 12) Transesterified Ethoxylated Vegetable Oils

These include transesterified ethoxylated vegetable oils such as those obtained by reacting various natural vegetable oils (for example, corn oil, maize oil, castor oil, kernel oil, almond oil, ground nut oil, olive oil, soybean oil, sunflower oil, safflower oil and palm oil, or mixtures thereof) with polyethylene glycols that have an average molecular weight of from 200 to 800, in the presence of an appropriate catalyst. These procedures are described in United States patent specification U.S. Pat. No. 3,288,824. Transesterified ethoxylated corn oil is particularly preferred.
Transesterified ethoxylated vegetable oils are known and are commercially available under the trade name Labrafil® (H. Fiedler, loc. cit., vol 2, page 994). Examples are Labrafil® M 2125 CS (obtained from corn oil and having an acid value of less than about 2, a saponification value of 155 to 175, an HLB value of 3 to 4, and an iodine value of 90 to 110), and Labrafil® M 1944 CS (obtained from kernel oil and having an acid value of about 2, a saponification value of 145 to 175 and an iodine value of 60 to 90). Labrafil® M 2130 CS (which is a transesterification product of a C₁₂₋₁₈ glyceride and polyethylene glycol and which has a melting point of about 35 to 40° C., an acid value of less than about 2, a saponification value of 185 to 200 and an iodine value of less than about 3) may also be used. The preferred transesterified ethoxylated vegetable oil is Labrafil® M 2125 CS which can be obtained, for example, from Gattefossé, Saint-Priest Cedex, France.

### 13) Sorbitan Fatty Acid Esters

Such esters include e.g. sorbitan mono C₁₂₋₁₈ fatty acid esters, or sorbitan tri C₁₂₋₁₈ fatty acid esters are commercially available under the trade mark Span® from e.g. uniqema. An especially preferred product of this class is e.g. Span® 20 (sorbitan monolaurate) or Span® 80 (sorbitan monooleate) (Fiedler, loc. cit., 2, p. 1572; Handbook of Pharmaceutical Excipients, 3^{rd} Edition, American Pharmaceutical Association and Pharmaceutical Press, loc. cit., page 511).

### 14) Esterified Compounds of Fatty Acid and Primary Alcohols

These include esterified compounds of fatty acid having 8 to 20 carbon atoms and primary alcohol having 2 to 3 carbon atoms, for example, isopropyl myristate, isopropyl palmitate, ethyl linoleate, ethyl oleate, ethylmyristate etc., with an esterified compound of linoleic acid and ethanol being particularly preferable, also isopropylmyristat and isopropylpalmitat.
   15) Glycerol Triacetate or (1,2,3)-triacetin
This is obtained by esterifying glycerin with acetic anhydride. Glycerol triacetate is commercially available as, e.g. Priacetin® 1580 from Unichema International, or as Eastman™ Triacetin from Eastman, or from Courtaulds Chemicals Ltd. Glycerol triacetate exhibits the following additional characterising data: molecular weight 218.03, D.^{20,3} 1.159-1.163, n_{D}²⁰ 1.430-1.434, water content max. 0.2%, viscosity)(25°) 17.4 mPa s, acid value max. 0.1, saponification value of about 766-774, triacetin content 97% min. (H. Fiedler, loc. cit., vol 2, page 1720, manufacturer information).

### 16) Acetyl Triethyl Citrate

This is obtained by esterification of citric acid and ethanol, followed by acetylation with acetic anhydride, respectively. Acetyl triethyl citrate is commercially available, e.g. under the trade name Citroflex® A-2, from e.g. Morflex Inc.

### 17) Tributylcitrate or Acetyl Tributyl Citrate

### 18) Polyglycerol Fatty Acid Esters

These have for example from 2 to 10, e.g. 6 glycerol units. The fatty acid constituent can include both saturated and unsaturated fatty acids having a chain length of from e.g. C₈-C₁₈. Particularly suitable is e.g. Plurol Oleique CC497 from Gattefossé, having a saponification value of 133-155 and a saponification value of 196-244. Further suitable polyglycerol fatty acid esters include diglyceryl monooleate (DGMO) and Hexaglyn-5-O, as known and commercially available from e.g. Nikko Chemicals Co., Ltd.

### 19) PEG-Fatty Alcohol Ether

This includes Brij 30™ polyoxyethylene(4) lauryl ether.

### 20) Fatty Alcohols and Fatty Acids

Fatty acids can be obtained by hydrolysing various animal and vegetable fats or oils, such as olive oil, followed by separation of the liquid acids. The fatty acid/alcohol constituent can include both saturated and mono- or di-unsaturated fatty acids/alcohols having a chain length of from e.g. C₆-C₂₀. Particularly suitable are, e.g. oleic acid, oleyl alcohol, linoleic acid, capric acid, caprylic acid, caproic acid, tetradecanol, dodecanol, or decanol. Oleyl alcohol is commercially available under the trade mark HD-Eutanol® V from e.g. Henkel KGaA. Oleyl alcohol exhibits the following additional characterising data: acid value max 0.1, hydroxy value of about 210, iodine value of about 95, saponification value max 1, D.²⁰ about 0.849, n_{D}²⁰ 1.462, molecular weight 268, viscosity)(20°) about 35 mPa s (manufacturer information). Oleic acid exhibits the following additional characterising data: molecular weight 282.47, D.²⁰ 0.895, n_{D}²⁰ 1.45823, acid value 195-202, iodine value 85-95, viscosity) (25°) 26 mPa s (H. Fiedler, loc. cit., volume 2, page 1238).
   21) Tocopherol and its Derivatives (e.g. Acetate)
These include Coviox T-70, Copherol 1250, Copherol F-1300, Covitol 1360 and Covitol 1100.

### 22) Pharmaceutically Acceptable Oils

Alternatively the lipophilic component comprises e.g. a pharmaceutically acceptable oil, preferably with an unsaturated component such as a vegetable oil.

### 23) Alkylene Polyol Ethers or Esters

These include C₃₋₅alkylene triols, in particular glycerol, ethers or esters. Suitable C₃₋₅alkylene triol ethers or esters include mixed ethers or esters, i.e. components including other ether or ester ingredients, for example transesterification products of C₃₋₅alkylene triol esters with other mono-, di- or poly-ols. Particularly suitable alkylene polyol ethers or esters are mixed C₃₋₅alkylene triol/poly-(C₂₋₄alkylene) glycol fatty acid esters, especially mixed glycerol/polyethylene- or polypropylene-glycol fatty acid esters.
Especially suitable alkylene polyol ethers or esters include products obtainable by transesterification of glycerides, e.g. triglycerides, with poly-(C₂₋₄alkylene) glycols, e.g. poly-ethylene glycols and, optionally, glycerol. Such transesterification products are generally obtained by alcoholysis of glycerides, e.g. triglycerides, in the presence of a poly-(C₂₋₄alkylene) glycol, e.g. polyethylene glycol and, optionally, glycerol (i.e. to effect transesterification from the glyceride to the poly-alkylene glycol/glycerol component, i.e. via poly-alkylene glycolysis/glycerolysis).
In general such reaction is effected by reacting the indicated components (glyceride, polyalkylene glycol and, optionally, glycerol) at elevated temperature under an inert atmosphere with continuous agitation.
Preferred glycerides are fatty acid triglycerides, e.g. (C₁₀₋₂₂fatty acid) triglycerides, including natural and hydrogenated oils, in particular vegetable oils. Suitable vegetable oils include, for example, olive, almond, peanut, coconut, palm, soybean and wheat germ oils and, in particular, natural or hydrogenated oils rich in (C₁₂₋₁₈fatty acid) ester residues. Preferred polyalkylene glycol materials are polyethylene glycols, in particular polyethylene glycols having a molecular weight of from ca. 500 to ca. 4,000, e.g. from ca. 1,000 to ca. 2,000.
Suitable alkylene polyol ethers or esters include mixtures of C₃₋₅alkylene triol esters, e.g. mono-, di- and tri-esters in variable relative amount, and poly (C₂₋₄alkylene) glycol mono- and di-esters, together with minor amounts of free C₃₋₅alkylene triol and free poly-(C₂₋₅alkylene) glycol. As hereinabove set forth, the preferred alkylene triol moiety is glyceryl; preferred polyalkylene glycol moieties include polyethylene glycol, in particular having a molecular weight of from ca. 500 to ca. 4,000; and preferred fatty acid moieties will be C₁₀₋₂₂fatty acid ester residues, in particular saturated C₁₀₋₂₂fatty acid ester residues.
Particularly suitable alkylene polyol ethers or esters include transesterification products of a natural or hydrogenated vegetable oil and a polyethylene glycol and, optionally, glycerol; or compositions comprising or consisting of glyceryl mono-, di- and tri-C₁₀₋₂₂fatty acid esters and polyethylene glycol mono- and di-C₁₀₋₂₂fatty esters (optionally together with, e.g. minor amounts of free glycerol and free polyethylene glycol).
Preferred vegetable oils, polyethylene glycols or polyethylene glycol moieties and fatty acid moieties in relation to the above definitions are as hereinbefore set forth.
Particularly suitable alkylene polyol ethers or esters as described above for use in the present invention include those commercially available under the trade name Gelucire® from e.g. Gattefossé, in particular the products:
a) Gelucire® 33/01, which has an m.p.=ca. 33-37° C. and a saponification value of ca. 230-255;
b) Gelucire® 39/01, m.p.=ca. 37.5-41.5° C., saponification v.=ca. 225-245;
c) Gelucire® 43/01, m.p.=ca. 42-46° C., saponification v.=ca. 220-240;
Products (a) to (c) above all have an acid value of maximum of 3. The compositions of the invention may include mixtures of such ethers or esters.

### 24) Hydrocarbons

These include e.g. squalene, available from e.g. Nikko Chemicals Co., Ltd.

### 25) Ethylene Glycol Esters

These include Monthyle® (ethylene glycol monostearate), available from e.g. Gattefossé.

### 26) Pentaerythriol Fatty Acid Esters and Polyalkylene Glycol Ethers

These include, for example pentaerythrite-dioleate, -distearate, -monolaurate, -polyglycol ether, and -monostearate as well as pentaerythrite-fatty acid esters (Fiedler, loc. cit., 2, p. 1288-1290, incorporated herein by reference).

Some of these, e.g. (1-3, 5-6, 8-9, 12-13, 19), display surfactant-like behaviour and may also be termed co-surfactants.

The lipophilic component preferably comprises 5 to 85% by weight of the composition of the invention, e.g. 10 to 85%; preferably about 15 to 60% by weight.

The liquid or semi-solid fill material of the present invention may additionally comprise a hydrophilic component.

### Suitable Hydrophilic Compounds Include:

### 1) Polyethylene Glycol Glyceryl C₆-C₁₀ Fatty Acid Esters

The fatty acid ester may include mono and/or di and/or tri fatty acid esters. It optionally includes both saturated and unsaturated fatty acids having a chain length of from e.g. C₈-C₁₀. The polyethylene glycols may have e.g. from 5 to 10 [CH₂—CH₂—O] units, e.g. 7 units. A particularly suitable fatty acid ester is polyethylene glycol (7) glyceryl monococoate, which is commercially available, e.g. under the trade name Cetiol® HE, e.g. from Henkel KGaA. Cetiol® HE has a D. (20° of 1.05, an acid value of less than 5, a saponification value of about 95, a hydroxyl value of about 180 and an iodine value of less than 5 (H. Fiedler, loc. cit., vol 1, page 409) or Lipestrol E-810.

### 2) N-alkylpyrrolidone

Particularly suitable is, e.g. N-Methyl-2-pyrrolidone, e.g. as commercially available under the trade name Pharmasolve™, from e.g. International Specialty Products (ISP). N-methylpyrrolidone exhibits the following additional characterising data: molecular weight 99.1, D.²⁵ 1.027-1.028, purity (as area % by GC) (including Methyl Isomers) 99.85% min (H. Fiedler, loc. cit., vol 2, page 1303, manufacturer information).

### 3) Benzyl Alcohol

This is commercially available from e.g. Merck or may be obtained by distillation of benzyl chloride with potassium or sodium carbonate. Benzyl alcohol exhibits the following additional characterising data: molecular weight 108.14, D. 1.043-1.049, n_{D} 1.538-1.541. (H. Fiedler, loc. cit., vol 1, page 301).

### 4) Triethyl Citrate

It is obtained esterifying citric acid and ethanol. Triethyl citrate is commercially available, e.g. under the trade names Citroflex® 2, or in a pharmaceutical grade under the name TEC-PG/N, from e.g. Morflex Inc. Particularly suitable is triethyl citrate which has molecular weight of 276.3, a specific gravity of 1.135-1.139, a refractive index of 1.439-1.441, a viscosity)(25° of 35.2 mPa s, assay (anhydrous basis) 99.0-100.5%, water max. 0.25% (Fiedler, H. P., loc. cit., vol 1, page 446).

Other suitable hydrophilic compounds include transcutol (C₂H₅—[O—(CH₂)₂]₂—OH), glycofurol (also known as tetrahydrofurfuryl alcohol polyethylene glycol ether), 1,2-propylene glycol, dimethylisosorbide (e.g. Arlasolve from Uniqema), polyethylene glycol (such as 200, 300, 400, 600, etc.), triethylenglycol, ethylacetate, and ethyllactate.

The hydrophilic component may comprise 5 to 60% by weight of the composition of the invention, e.g. 5 to 50%; preferably 5 to 40% by weight

The hydrophilic component may comprise a mixture of two or more hydrophilic components. The ratio of main hydrophilic component to hydrophilic co-component is typically from about 0.5:1 to about 2:1.

The liquid fill material of the compositions of the present invention may preferably contain one or more surfactants to reduce the interfacial tension thereby providing thermodynamic stability.

Surfactants may be complex mixtures containing side products or unreacted starting products involved in the preparation thereof, e.g. surfactants made by polyoxyethylation may contain another side product, e.g. polyethylene glycol. Each surfactant preferably has a hydrophilic-lipophilic balance (HLB) value of 8 to 17, especially 10 to 17. The HLB value is preferably the mean HLB value.

### Suitable Surfactants Include:

### 1) Reaction Products of a Natural or Hydrogenated Castor Oil and Ethylene Oxide

The natural or hydrogenated castor oil may be reacted with ethylene oxide in a molar ratio of from about 1:35 to about 1:60, with optional removal of the polyethylene-glycol component from the products. Various such surfactants are commercially available. Particularly suitable surfactants include polyethyleneglycol-hydrogenated castor oils available under the trade name Cremophor®; Cremophor® RH 40, which has a saponification value of about 50 to 60, an acid value less than about 1, a water content (Fischer) less than about 2%, an n_{D}⁶⁰ of about 1.453-1.457 and an HLB of about 14-16; and Cremophor® RH 60, which has a saponification value of about 40-50, an acid value less than about 1, an iodine value of less than about 1, a water content (Fischer) of about 4.5-5.5%, an n_{D}⁶⁰ of about 1.453-1.457 and an HLB of about 15 to 17.

An especially preferred product of this class is Cremophor® RH40. Other useful products of this class are available under the trade names Nikkol® (e.g. Nikkol® HCO-40 and HCO-60), Mapeg® (e.g. Mapeg® CO-40h), Incrocas® (e.g. Incrocas® 40), Tagat® (for example polyoxyethylene-glycerol-fatty acid esters e.g. Tagat® RH 40) and Simulsol OL-50 (PEG-40 castor oil, which has a saponification value of about 55 to 65, an acid value of max. 2, an iodine value of 25 to 35, a water content of max. 8%, and an HLB of about 13, available from Seppic). These surfactants are further described in Fiedler loc. cit.

Other suitable surfactants of this class include polyethyleneglycol castor oils such as that available under the trade name Cremophor® EL, which has a molecular weight (by steam osmometry) of about 1630, a saponification value of about 65 to 70, an acid value of about 2, an iodine value of about 28 to 32 and an n_{D}²⁵ of about 1.471.

### 2) Polyoxyethylene-Sorbitan-Fatty Acid Esters

These include mono- and tri-lauryl, palmityl, stearyl and oleyl esters of the type known and commercially available under the trade name Tween® (Fiedler, loc. cit. p. 1754 ff) from Uniqema including the products:
Tween® 20 [polyoxyethylene(20)sorbitanmonolaurate],
Tween® 21 [polyoxyethylene(4)sorbitanmonolaurate],
Tween® 40 [polyoxyethylene(20)sorbitanmonopalmitate],
Tween® 60 [polyoxyethylene(20)sorbitanmonostearate],
Tween® 65 [polyoxyethylene(20)sorbitantristearate],
Tween® 80 [polyoxyethylene(20)sorbitanmonooleate],
Tween® 81 [polyoxyethylene(5)sorbitanmonooleate], and
Tween® 85 [polyoxyethylene(20)sorbitantrioleate].
Especially preferred products of this class are Tween® 20 and Tween® 80.

### 3) Polyoxyethylene Fatty Acid Esters

These include polyoxyethylene stearic acid esters of the type known and commercially available under the trade name Myrj® from Uniqema (Fiedler, loc. cit., 2, p. 1166). An especially preferred product of this class is Myrj® 52 having a D²⁵ of about 1.1, a melting point of about 40 to 44° C., an HLB value of about 16.9, an acid value of about 0 to 1 and a saponification no. of about 25 to 35.

### 4) Polyoxyethylene-Polyoxypropylene Co-Polymers and Block Co-Polymers or Poloxamers

These include the type known and commercially available under the trade names Pluronic® and Emkalyx® (Fiedler, loc. cit., 2, p. 1329). An especially preferred product of this class is Pluronic® F68 (poloxamer 188) from BASF, having a melting point of about 52° C. and a molecular weight of about 6800 to 8975. A further preferred product of this class is Synperonic® PE L44 (poloxamer 124) from Uniqema.

### 5) Polyoxyethylene Mono Esters of a Saturated C₁₀ to C₂₂

These include C₁₈ substituted e.g. hydroxy fatty acid; e.g. 12 hydroxy stearic acid PEG ester, e.g. of PEG about e.g. 600-900 e.g. 660 Daltons MW, e.g. Solutol® HS 15 from BASF, Ludwigshafen, Germany. According to the BASF technical leaflet MEF 151 E (1986) comprises about 70% polyethoxylated 12-hydroxystearate by weight and about 30% by weight unesterified polyethylene glycol component. Solutol HS 15 has a hydrogenation value of 90 to 110, a saponification value of 53 to 63, an acid number of maximum 1, and a maximum water content of 0.5% by weight.

### 6) Polyoxyethylene Alkyl Ethers

These include polyoxyethylene glycol ethers of C₁₂ to C₁₈ alcohols, e.g. Polyoxyl 2-, 10- or 20-cetyl ether or Polyoxyl 23-lauryl ether, or polyoxyl 20-oleyl ether, or Polyoxyl 2-, 10-, 20- or 100-stearyl ether, as known and commercially available e.g. under the trade mark Brij® from Uniqema. An especially preferred product of this class is e.g. Brij® 35 (Polyoxyl 23 lauryl ether) or Brij® 98 (Polyoxyl 20 oleyl ether) (Fiedler, loc. cit., 1, pp. 326; Handbook of Pharmaceutical Excipients, loc. cit., page 407). Similarly suitable products include polyoxyethylene-polyoxypropylene-alkyl ethers, e.g. polyoxyethylene-polyoxypropylene-ethers of C₁₂ to C₁₈ alcohols, e.g. polyoxyethylen-20-polyoxypropylene-4-cetylether which is known and commercially available under the trade mark Nikkol PBC® 34, from e.g. Nikko Chemicals Co., Ltd. Polyoxypropylene fatty acid ethers, e.g. Acconon® E are also suitable.

### 7) Sodium Alkyl Sulfates and Sulfonates, and Sodium Alkyl Aryl Sulfonates

These include sodium lauryl sulfate, which is also known as sodium dodecyl sulfate and commercially available, e.g. under the trade name Texapon K12® from Henkel KGaA.

### 8) Water Soluble Tocopheryl Polyethylene Glycol Succinic Acid Esters (TPGS)

These include those with a polymerisation number ca 1000 or 400, e.g. available from Eastman Fine Chemicals Kingsport, Tex., USA.

### 9) Polyglycerol Fatty Acid Esters

These include those with e.g. from 10 to 20, e.g. 10 glycerol units. The fatty acid constituent may include both saturated and unsaturated fatty acids having a chain length of from e.g. C₈-C₁₈. Particularly suitable is e.g. decaglycerylmonolaurat or decaglycerylmonomyristat, as known and commercially available under the trade mark Decaglyn® 1-L or Decaglyn® 1-M or Decaglyn 1-0, respectively, from e.g. Nikko Chemicals C., Ltd (Fiedler, loc. cit., vol. 2, pp. 1359).

### 10) Alkylene Polyol Ethers or Esters

These include C₃₋₅alkylene triols, in particular glycerol, ethers or esters. Suitable C₃₋₅alkylene triol ethers or esters include mixed ethers or esters, i.e. components including other ether or ester ingredients, for example transesterification products of C₃₋₅alkylene triol esters with other mono-, di- or poly-ols. Particularly suitable alkylene polyol ethers or esters are mixed C₃₋₅alkylene triol/poly-(C₂₋₄alkylene) glycol fatty acid esters, especially mixed glycerol/polyethylene- or polypropylene-glycol fatty acid esters.
Especially suitable alkylene polyol ethers or esters include products obtainable by transesterification of glycerides, e.g. triglycerides, with poly-(C₂₋₄alkylene) glycols, e.g. poly-ethylene glycols and, optionally, glycerol.
Such transesterification products are generally obtained by alcoholysis of glycerides, e.g. triglycerides, in the presence of a poly-(C-₂₋₄alkylene) glycol, e.g. polyethylene glycol and, optionally, glycerol (i.e. to effect transesterification from the glyceride to the poly-alkylene glycol/glycerol component, i.e. via poly-alkylene glycolysis/gly-cerolysis). In general such reaction is effected by reacting the indicated components (glyceride, polyalkylene glycol and, optionally, glycerol) at elevated temperature under an inert atmosphere with continuous agitation.
Preferred glycerides are fatty acid triglycerides, e.g. (C₁₀₋₂₂fatty acid) triglycerides, including natural and hydrogenated oils, in particular vegetable oils. Suitable vegetable oils include, for example, olive, almond, peanut, coconut, palm, soybean and wheat germ oils and, in particular, natural or hydrogenated oils rich in (C₁₂₋₁₈fatty acid) ester residues.
Preferred polyalkylene glycol materials are polyethylene glycols, in particular polyethylene glycols having a molecular weight of from ca. 500 to ca. 4,000, e.g. from ca. 1,000 to ca. 2,000.

Suitable alkylene polyol ethers or esters include mixtures of C₃₋₅alkylene triol esters, e.g. mono-, di- and tri-esters in variable relative amount, and poly (C₂₋₄alkylene) glycol mono- and di-esters, together with minor amounts of free C₃₋₅alkylene triol and free poly-(C₂₋₅alkylene) glycol. As hereinabove set forth, the preferred alkylene triol moiety is glyceryl; preferred polyalkylene glycol moieties include polyethylene glycol, in particular having a molecular weight of from ca. 500 to ca. 4,000; and preferred fatty acid moieties will be C₁₀₋₂₂fatty acid ester residues, in particular saturated C₁₀₋₂₂fatty acid ester residues.
Particularly suitable alkylene polyol ethers or esters include transesterification products of a natural or hydrogenated vegetable oil and a polyethylene glycol and, optionally, glycerol; or compositions comprising or consisting of glyceryl mono-, di- and tri-C₁₀₋₂₂fatty acid esters and polyethylene glycol mono- and di-C₁₀₋₂₂fatty esters (optionally together with, e.g. minor amounts of free glycerol and free polyethylene glycol).
Preferred vegetable oils, polyethylene glycols or polyethylene glycol moieties and fatty acid moieties in relation to the above definitions are as hereinbefore set forth.
Particularly suitable alkylene polyol ethers or esters as described above for use in the present invention include those commercially available under the trade name Gelucire® from e.g. Gattefossé, in particular the products:
a) Gelucire® 44/14, m.p.=ca. 42.5-47.5° C., saponification v.=ca. 79-93;
b) Gelucire® 50/13, m.p.=ca. 46-51° C., saponification v.=ca. 67-81;
Products (a) to (b) above all have an acid value of maximum of 2.
Alkylene polyol ethers or esters having an iodine value of maximum 2 are generally preferred. The compositions of the invention may include mixtures of such ethers or esters.
Gelucire® products are inert semi-solid waxy materials with amphiphilic character. They are identified by their melting point and their HLB value. Most Gelucire® grades are saturated polyglycolised glycerides obtainable by polyglycolysis of natural hydrogenated vegetable oils with polyethylene glycols. They are composed of a mixture of mono-, di- and tri-glycerides and mono- and di-fatty acid esters of polyethylene glycol. Particularly suitable is Gelucire® 44/14 which has a nominal melting point of 44° C. and an HLB of 14. It is obtained by reacting hydrogenated palm kernels and/or hydrogenated palm oils with polyethylene glycol 1500. It consists of approximately 20% mono-, di- and triglycerides, 72% mono- and di-fatty acid esters of polyethylene glycol 1500 and 8% of free polyethylene glycol 1500. The fatty acid distribution for Gelucire® 44/14 is as follows: 4-10 C₈, 3-9 C₁₀, 40-50 C₁₂, 14-24 C₁₄, 4-14 C₁₆, 5-15 C₁₈. Gelucire® 44/14 exhibits the following additional characterising data: acid value of max. 2, iodine value of max. 2, saponification value of 79-93, hydroxyl value of 36-56, peroxide value of max. 6, alkaline impurities max. 80, water content max. 0.50, free glycerol content max. 3, monoglycerides content 3.0-8.0. (H. Fiedler, loc. cit., vol 1, page 773; manufacturer information).

### 11) Polyethylene Glycol Glyceryl Fatty Acid Esters

The fatty acid ester may include mono and/or di and/or tri fatty acid ester. The fatty acid constituent may include both saturated and unsaturated fatty acids having a chain length of from e.g. C₁₂-C₁₈. The polyethylene glycols may have e.g. from 10 to 40 [CH₂—CH₂—O] units, e.g. 15 or 30 units. Particularly suitable is polyethylene glycol (15) glyceryl monostearat which is commercially available, e.g. under the trade name TGMS®-15, e.g. from Nikko Chemicals Co., Ltd. Other suitable glyceryl fatty acid esters include polyethylene glycol (30) glyceryl monooleate which is commercially available, e.g. under the trade name Tagat® 0, e.g. from Goldschmidt (H. Fiedler, loc. cit., vol. 2, p. 1650), and Tagat O2 (polytheylene glycol (20) glycerol monooleate, as well as Tagat L (polytheylene glycol (30) glycerol monolaurate) and Tagat L2 (polytheylene glycol (20) glycerol monolaurate), all e.g. from Goldschmidt (H. Fiedler, loc. cit., vol. 2, p. 1650). A further suitable polyethylene glycol glyceryl fatty acid ester is Tagat TO.

### 12) Sterols and Derivatives Thereof.

These include cholesterols and derivatives thereof, in particular phytosterols, e.g. products comprising sitosterol, campesterol or stigmasterol, and ethylene oxide adducts thereof, for example soya sterols and derivatives thereof, e.g. polyethylene glycol sterols, e.g. polyethylene glycol phytosterols or polyethylene glycol soya sterols. The polyethylene glycols may have e.g. from 10 to 40 [CH₂—CH₂—O] units, e.g. 25 or 30 units. Particularly suitable is polyethylene glycol (30) phytosterol which is commercially available, e.g. under the trade name Nikkol BPS®-30, e.g. from Nikko Chemicals Co., Ltd. Further suitable is polyethylene glycol (25) soya sterol which is commercially available, e.g. under the trade name Generol® 122 E 25, e.g. from Henkel (H. Fiedler, loc. cit., vol. 1, p. 779).

### 13) Transesterified, Polyoxyethylated Caprylic-Capric Acid Glycerides

These include those that are commercially available under the trade name Labrasol® from e.g. Gattefossé. Labrasol® has an acid value of max. 1, a saponification value of 90-110, and an iodine value of max. 1 (H. Fiedler, loc. cit., vol 2, page 995).

### 14) Sugar Fatty Acid Esters

These include those of C₁₂-C₁₈ fatty acids, e.g. sucrose monolaurate, e.g. Ryoto L-1695®, which is commercially available from e.g. Mitsubishi-Kasei Food Corp., Tokyo, Japan.

### 15) PEG Sterol Ethers

These include those having, e.g. from 5 to 35 [CH₂—CH₂—O] units, e.g. 20 to 30 units, e.g. Solulan® C24, which is commercially available from e.g. Amerchol.

### 16) Dioctylsodiumsulfosuccinate

This is commercially available under the trade mark Aerosol OT® from e.g. American Cyanamid Co. (Fiedler, loc. cit., 1, p. 164), or di-[2-ethylhexyl]-succinate.

### 17) Phospholipids

These include in particular lecithins (Fiedler, loc. cit., volume 2, p. 910, 1030). Suitable lecithins include, in particular, soya bean lecithins.

### 18) Salts of Fatty Acids, Fatty Acid Sulfates and Sulfonates

These include those of e.g. C₆-C₁₈, fatty acids, -fatty acid sulfates and sulfonates, as known and commercially available from e.g. Fluka.

### 19) Salts of Acylated Amino Acids

These include those of C₆-C₁₈, acylated amino acids, e.g. sodium lauroyl sarcosinate, which is commercially available from e.g. Fluka.
   20) Medium or Long-Chain Alkyl, e.g. C₆-C₁₈, Ammonium Salts
These include C₆-C₁₈ acylated amino acids e.g. cetyl trimethyl ammonium bromide, which is commercially available from e.g. E. Merck AG.

The surfactant may comprise 5 to 90% by weight of the composition of the invention; preferably 10 to 85% by weight, more preferably 15 to 60% by weight.

The liquid fill material of the compositions of the present invention may optionally contain co-solvents to reduce the interfacial tension thereby providing thermodynamic stability. Suitable co-solvents include lower alkanols such as ethanol and transcutol. As a result the risk of active agent precipitation following encapsulation procedures may be reduced and storage characteristics may be improved. Thus the shelf life stability may be extended by employing ethanol or some other such co-component as an additional ingredient of the composition. The ethanol may comprise 0 to 60% by weight of the composition; preferably 5 to about 30% by weight and more preferably about 5 to 20% by weight.

In another aspect the invention provides a composition wherein the fill material is in the form of a microemulsion preconcentrate. The microemulsion preconcentrate may comprise a lipophilic component, a surfactant, a hydrophilic component and optionally a co-solvent.

In another aspect, the invention provides a process for the preparing a microemulsion preconcentrate containing an active agent, which process comprises:

bringing the active agent and a carrier comprising (1) a lipophilic component, (2) a surfactant, (3) a hydrophilic component, and optionally (4) a co-solvent into intimate admixture to form a spontaneously dispersible pharmaceutical composition;

The pharmaceutically acceptable acid may be dissolved in the hydrophilic component or, if present in the co-solvent and introduced in the mixture.

In a further aspect, the invention provides a process for the preparing a microemulsion containing an active agent, which process comprises:
(i) bringing the active agent and a carrier comprising (1) a lipophilic component, (2) a surfactant, (3) a hydrophilic component, and optionally (4) a co-solvent into intimate admixture to form a spontaneously dispersible pharmaceutical composition; and
(ii) diluting the spontaneously dispersible pharmaceutical composition in an aqueous medium to form the microemulsion.
The pharmaceutically acceptable acid may be dissolved in the hydrophilic component or, if present in the co-solvent and introduced in the mixture of step (i).

The active agent may be present in an amount by weight of up to about 20% by weight of the composition of the invention, e.g. from about 0.05% by weight. The active agent is preferably present in an amount of about 0.05 to about 15% by weight of the composition, more preferably in an amount of about 0.05 to about 10% by weight of the composition.

The fill material or the liquid fill material may be filled into hard gelatin capsules or soft gelatin capsules.

In another aspect the present invention provides a process for preparation of hard gelatin capsules. Hard gelatin capsules may be prepared according to any suitable process, e.g., as described in Remington: The Science and Practice of Pharmacy, 19^{th} Ed., Mack Publishing Co. Easton, Pa., loc. cit. p 1642 to 1643. The pharmaceutically acceptable acid may be added to the gelatin mixture.

In a further aspect the present invention provides a process for preparation soft gelatin capsules. Soft gelatin capsules according the present invention may be prepared according to any suitable, e.g., according to the plate-process or the rotary-die process as, e.g., described in Remington: The Science and Practice of Pharmacy, 19^{th} Ed., Mack Publishing Co. Easton, Pa., loc. cit. p 1646 to 1647. The pharmaceutically acceptable acid may be added to the gelatin mixture.

In a further aspect the present invention provides the use of a pharmaceutically acceptable acid to avoid pellicle formation in a pharmaceutical composition in the form of a gelatin capsule.

Dosage forms of the present invention are useful in the treatment and prevention of a wide range of disorders as indicated by the active agents mentioned above. Dosage forms of the present invention, e.g. gelatine capsules, comprising a pharmaceutically acceptable acid, e.g., citric acid in the shell or in the fill material or in the shell and in the fill are well tolerated by the gastrointestinal tract. The prevention of cross-linking and pellicle formation avoids a reduction of the dissolution rate and the therefore reduced bioavailability and any inconvenience which might be related to reduced bioavailability of an active agent.

Following is a description by way of example only of processes and compositions of the invention.

### EXAMPLES

The formulations of Table 1 are manufactured and subsequently filled into soft gelatin capsules with the same composition (gelatin, propylene glycol, glycerol). Composition A does not comprise a pharmaceutically acceptable acid, Composition B does comprise a pharmaceutically acceptable acid (citric acid) and Composition C does comprise a pharmaceutically acceptable acid (phosphoric acid)

TABLE 1
Composition of formulations

| | Composition A | Composition B | Composition C |
|---|---|---|---|
| | [mg] | [mg] | [mg] |
| Macrogol-Glycerol- | 223.25 | 223.25 | 223.25 |
| hydroxystearate | | | |
| Corn oil glycerides | 179.00 | 179.00 | 179.00 |
| Propylene glycol | 50.0 | 50.0 | 50.0 |
| Ethanol | 50.0 | 50.0 | 50.0 |
| Citric acid | — | 2.5 | |
| Diphosphoric | | | 1.0 |
| acid 85% | | | |
| Drug substance (7-t- | 0.25 | 0.25 | 0.25 |
| butoxyiminomethyl- | | | |
| campothecin) | | | |

The capsules are stored at 5° C. (control) and 40° C. at 75% RH (relative humidity) over 1.4 M (months), 2.3 M (months) and 12M (months). The in vitro dissolution rate (% drug dissolved per minute) of the poorly soluble drug substance 7-t-butoxyiminomethyl-campothecin is tested in a standard dissolution tester (Paddle apparatus). As illustrated in FIG. 1 composition A shows a strong pellicle formation when stored at 40° C. at 75% RH (relative humidity) resulting in no release of the drug substance after 1.4 M (months). The control of composition A stored at 5° C. does not show a reduced drug release. Composition B and C do not show pellicle formation resulting in a drug release which is the same after storage at 5° C. (control) and at 40° C./75% RH as illustrated in FIG. 2 after 2.4 M (months) and in FIG. 3 after 3 M (months). Composition B was further tested after 12M and does not show pellicle formation at elevated temperatures 30° C./65% RH as illustrated in FIG. 4.

### BRIEF DESCRIPTION OF THE DRAWINGS

FIG. 1 shows the dissolution rate of composition A after storage at 5° C. (control) and 40° C./75% RH for 1.4 M.

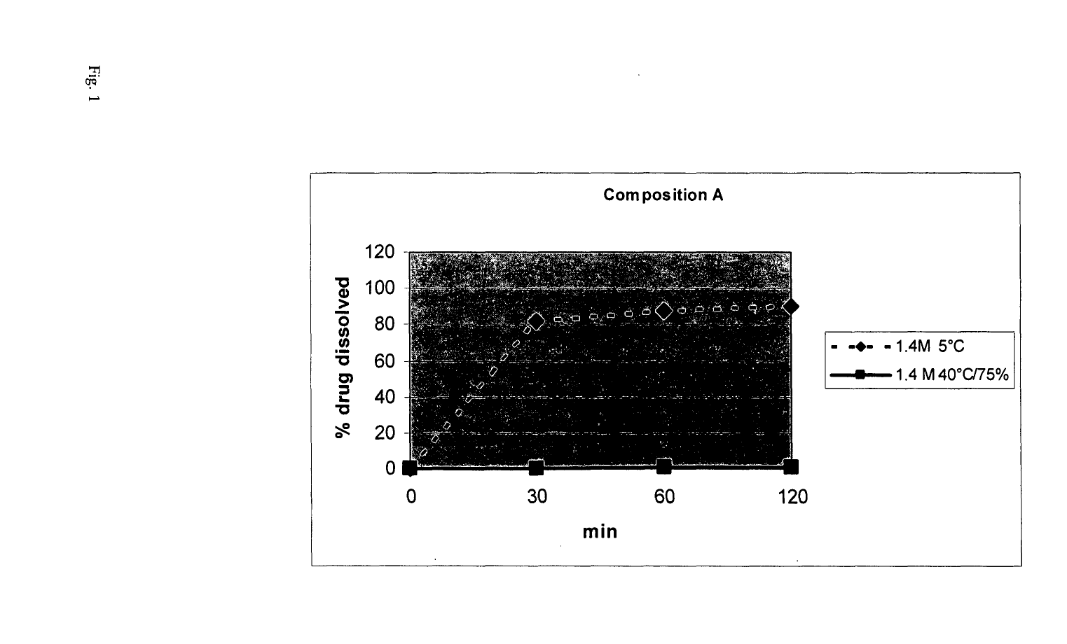

FIG. 2 shows the dissolution rate of composition B after storage at 5° C. (control) and 40° C./75% RH for 2.3 M.

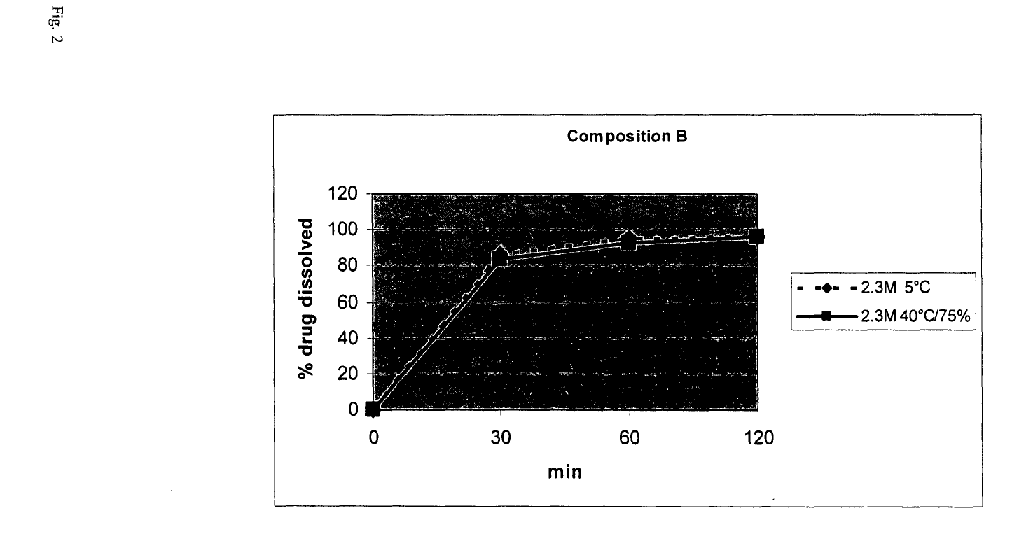

FIG. 3 shows the dissolution rate of composition C after storage at 5° C. (control) and 40° C./75% RH for 3 M.

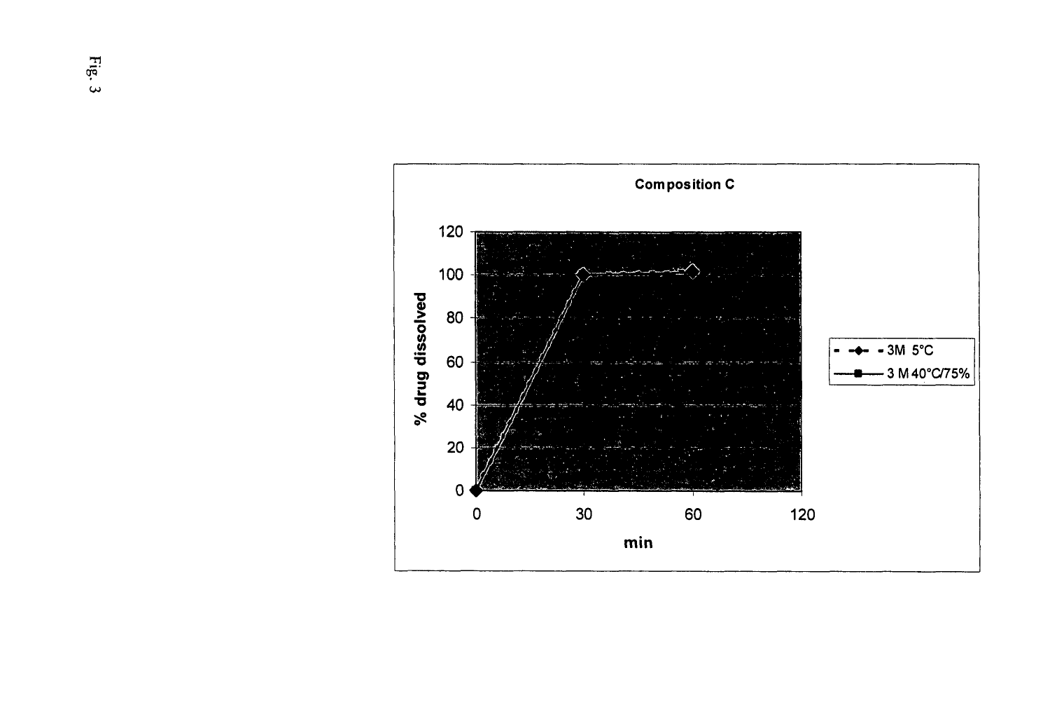

FIG. 4. shows the dissolution rate of composition B after storage at 5° C. (control) and 30° C./65% RH for 12 M.

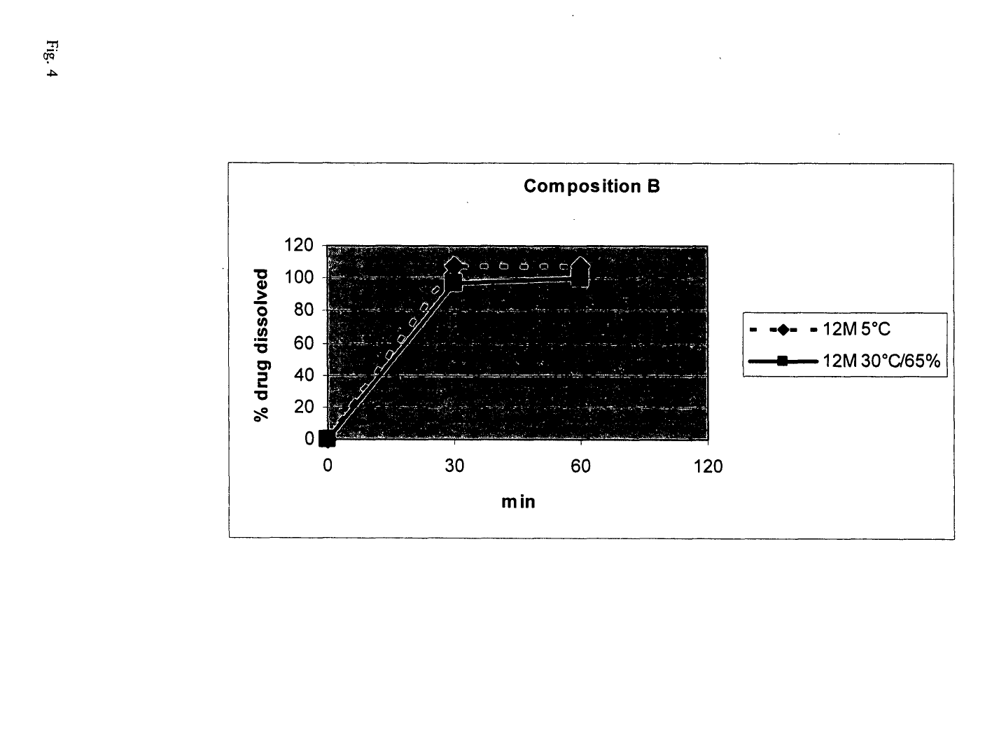

1. A pharmaceutical composition in the form of a gelatin capsule comprising a pharmaceutically acceptable acid.

2. The pharmaceutical composition according to claim 1 comprising a capsule shell and fill material.

3. The pharmaceutical composition according to claim 1 wherein the pharmaceutically acceptable acid is added to the fill material.

4. The pharmaceutical composition according to claim 3 wherein the pharmaceutically acceptable acid migrates from the fill material to the capsule shell.

5. The composition according to claim 3 wherein the pharmaceutically acceptable acid is selected from fumaric acid, phosphoric acid, malic acid, ascorbic acid, tartaric acid, malonic acid, glucuronic acid and citric acid.

6. The composition according to claim 5 wherein the pharmaceutically acceptable acid is citric acid or phosphoric acid.

7. The composition of claim 1 wherein the pharmaceutically acceptable acid is present in an amount effective to inhibit pellicle formation of the gelatin capsule.

8. The composition according to claim 7 wherein the pharmaceutically acceptable acid is present in an amount of 0.01% to 20% of the total weight of the fill material.

9. The composition according to claim 8 wherein the pharmaceutically acceptable acid is present in an amount of 0.1% to 2% of the total weight of the fill material.

10. The composition according to claim 7 wherein the pharmaceutically acceptable acid is present in amount of 0.01% to 20% of the total weight of the capsule shell.

11. The composition according to claim 10 wherein the pharmaceutically acceptable acid is present in an amount of 0.1% to 2% of the total weight of the capsule shell.

12. The composition according to claim 2 wherein the fill material is semi-liquid or liquid.

13. The composition according to claim 12 wherein the fill material is in the form of a microemulsion preconcentrate.

14. The composition according to claim 1 being a spontaneously dispersible pharmaceutical composition.

15. The composition according to claim 14 comprising 7-t-butoxyiminomethyl-campothecin.

16. The composition according to claim 14 comprising 7-t-butoxyiminomethyl-campothecin and a carrier medium comprising a lipophilic component, a surfactant, and a hydrophilic component.

17. The composition according to claim 16 further comprising a co-solvent.

18. The composition of claim 1 wherein the gelatin capsule is a soft gelatin capsule.

19. (canceled)
